(19) United States
(12) Patent Application Publication
(10) Pub. No.: US 2025/0177185 A1
(43) Pub. Date: Jun. 5, 2025

(54) INTERNAL OSTOMY BAG

(71) Applicant: BOSTON SCIENTIFIC SCIMED, INC., Maple Grove, MN (US)

(72) Inventors: Gonzalo Jose SAENZ VILLALOBOS, Alajuela (CR); John Thomas FAVREAU, Charlton, MA (US); Seamus Finbarr O’SHAUGHNESSY, Chelmsford, MA (US); Gian Franco LOO FUCHS, San Jose (CR); Kevin WOOD, Marlborough, MA (US)

(21) Appl. No.: 18/933,685

(22) Filed: Oct. 31, 2024

Related U.S. Application Data
(60) Provisional application No. 63/604,546, filed on Nov. 30, 2023.

(51) Int. Cl. A61F 5/448

(52) U.S. Cl. CPC A61F 5/448

(57) ABSTRACT

An ostomy bag device includes an expandable reservoir extending from a first end to a second end and defining a space therewithin. The device also includes first and second connectors. The first connector is connected to the first end and engages an opening extending through a wall at an end of an intestine so that intestinal contents flowing from the end of the intestine are received and collected within the reservoir. The second connector is connected to the second end and engages an exit location via which contents collected within the reservoir are to be drained to an exterior of a patient body. The second connector includes a control element movable between a closed configuration, in which contents of the reservoir are prevented from 10 flowing therepast, and an open configuration, in which contents of the reservoir are permitted to flow therepast to drain the reservoir.

### PRIORITY CLAIM

The present disclosure claims priority to U.S. Provisional Patent Application Ser. No. 63/604,546 filed Nov. 30, 2023; the disclosure of which is incorporated herewith by reference.

### FIELD

The present disclosure relates to ostomy devices and, in particular, relates to an internal ostomy bag configured to receive and collect intestinal contents therein.

### BACKGROUND

Ostomy procedures (e.g., colostomy, ileostomy) are performed for various patients with diseased or damaged bowels including, for example, colorectal cancer and diverticular disease or to treat issues in the urinary tract. Current ostomy procedures include re-routing a part of the colon (or small intestine) to drain outside of the body by bringing an end of the colon to the abdominal wall, where edges of the colon are stitched to the skin to form an opening called a stoma and/or urostomies in which a portion of an organ such as the small intestine is resected and coupled from the urinary tract (e.g., the ureters) to a stoma to permit the drainage of urine therethrough. Waste is then able to drain from the stoma into a bag or pouch attached to the abdomen. However, ostomy procedures often result in post-operative complications and/or patient discomfort including, for example, leakage, odor, skin irritation from attachment of the ostomy bag, and social anxiety.

### SUMMARY

The present disclosure claims priority to an ostomy bag device. The device includes an expandable reservoir and first and second connectors. The reservoir extends from a first end to a second end and defines a space therewithin. The first connector is connected to the first end of the reservoir and configured to engage an opening extending through a wall at an end of an intestine so that intestinal contents flowing from the end of the intestine are received and collected within the reservoir. The second connector is connected to the second end of the reservoir and configured to engage an exit location via which contents collected within the reservoir are to be drained to an exterior of a patient body. The second connector includes a control element movable between a closed configuration, in which contents of the reservoir are prevented from flowing therepast, and an open configuration, in which contents of the reservoir are permitted to flow therepast to drain the reservoir.

In an embodiment, the device further includes a sensor positioned along the reservoir and configured to detect a level of fill thereof.

In an embodiment, the sensor includes one of a pressure sensor and a deformation sensor.

In an embodiment, one of the first connector and the second connector includes a self-expanding mesh coil.

In an embodiment, the self-expanding mesh coil includes a first flange at a first end thereof and a second flange at a second end thereof, the first and second flanges configured to receive a portion of the wall of the intestine therebetween.

In an embodiment, one of the first and second connectors is connected to the first and second ends of the reservoir, respectively, via a tubing.

In an embodiment, the control element includes one of a valve and an artificial sphincter.

In an embodiment, the reservoir is formed of a biocompatible and expandable material.

In an embodiment, the biocompatible and expandable material includes silicone.

In an embodiment, the reservoir is deformable from an initial, unexpanded configuration toward an expanded configuration as the intestinal contents are received therein.

In addition, the present disclosure relates to a system. The system has a bag device including an expandable reservoir, a first end of the expandable reservoir configured to be connected to an end of an intestine via a first connector so that intestinal contents flowing from the end of the intestine are received and collected within the expandable reservoir, and a second end of the expandable reservoir configured to be connected to an exit location via a second connector so that the intestinal contents collected within the expandable reservoir are to be drained to an exterior of a patient body, the second connector including a control element movable between a closed configuration, in which contents of the reservoir are prevented from flowing therepast, and an open configuration, in which contents of the reservoir are permitted to flow therepast to drain the expandable reservoir.

The device also includes a delivery device extending longitudinally and including a channel extending therethrough, the bag device insertable through the channel to a target location within a user body.

In an embodiment, the system further includes a puncturing device configured to form an opening through a wall of an intestine to which one of the first and second connectors of the bag device is to be engaged.

In an embodiment, the bag device includes a sensor positioned along the reservoir, the sensor configured to detect a fill level of the reservoir.

In an embodiment, one of the first connector and the second connector includes a self-expanding mesh coil.

In an embodiment, the control element includes one of a valve and an artificial sphincter.

In addition, the present disclosure relates to a method of treating an intestine. The method engaging a first connector connected to a first end of a reservoir with an opening extending through a wall at an end of an intestine so that intestinal contents flowing from the end of the intestine are received and collected within the reservoir; and engaging a second connector connected to a second end of the reservoir to an exit location, the second connector including a control element movable between an open configuration, in which contents of the reservoir are permitted to flow through the control element, through the exit location, to an exterior of a user body, and a closed configuration, in which contents of the reservoir are prevented from flowing through the control element.

In an embodiment, the method further includes detecting a level of fill of the reservoir to determine whether to drain the reservoir.

In an embodiment, the exit location includes one of an anus and an opening extending through an abdominal wall of the user body.

In an embodiment, engaging the first connector with the opening extending through the wall of the intestine includes deploying a self-expanding mesh coil.

In an embodiment, the method further includes forming the opening through the wall at the end of the intestine via an electrocautery device.

### BRIEF DESCRIPTION

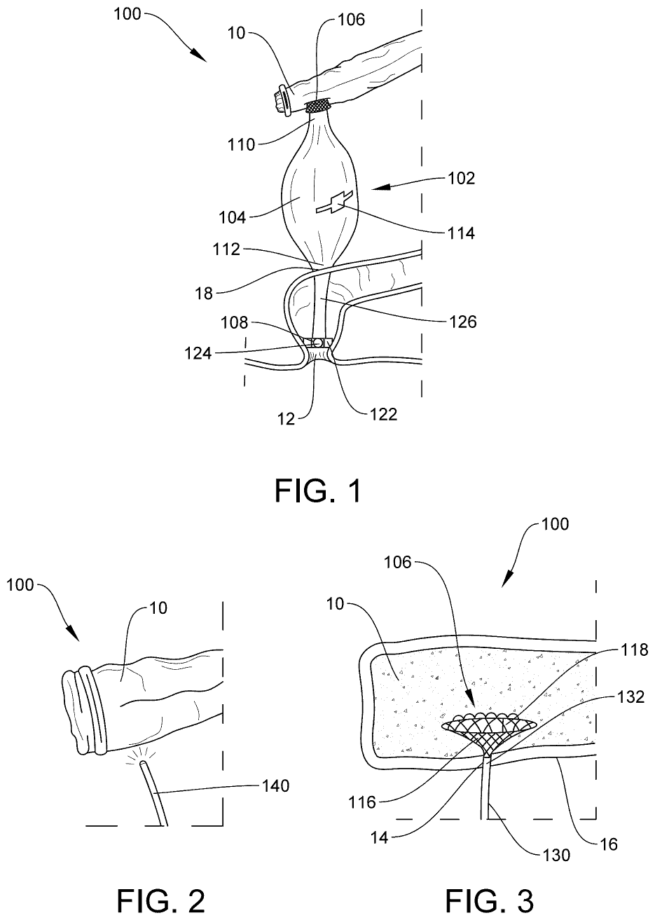

FIG. 1 shows a side view of a bag device of a system according to an exemplary embodiment of the present disclosure;

FIG. 2 shows a side view of a puncturing device of the system according to FIG. 1;

FIG. 3 shows a side view of a partially deployed connector at a first end of the bag device according to the system of FIG. 1;

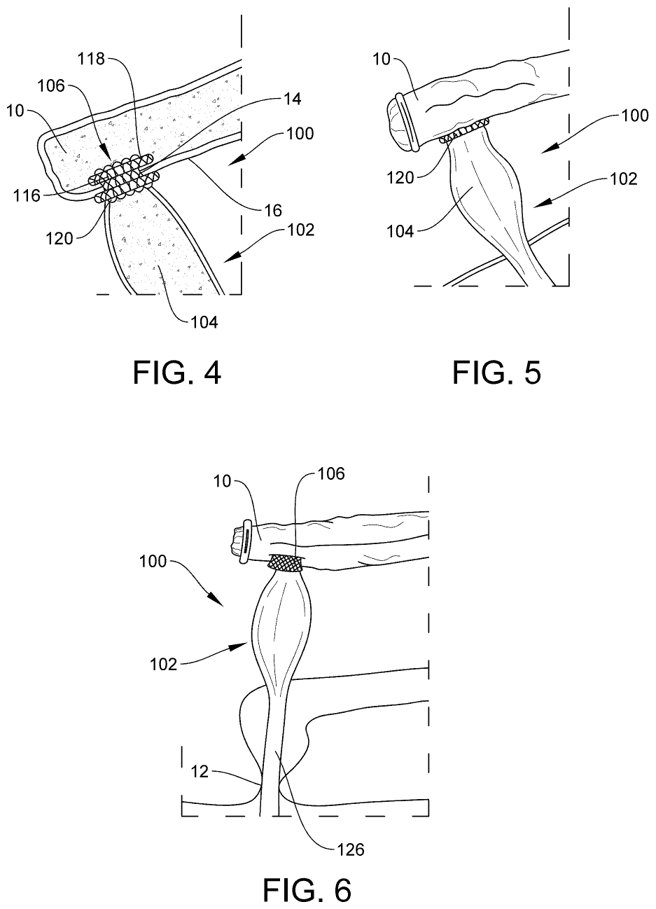

FIG. 4 shows a cross-sectional side view of the connector at the first end of the bag device engaging an end of an intestine, according to the system of FIG. 1;

FIG. 5 shows side view of the bag device, a second end thereof extending through a wall of a lumen to engage an exit location according to the system of FIG. 1;

FIG. 6 show a side view of a bag device positioned within a patient body according to an alternate embodiment of the present disclosure;

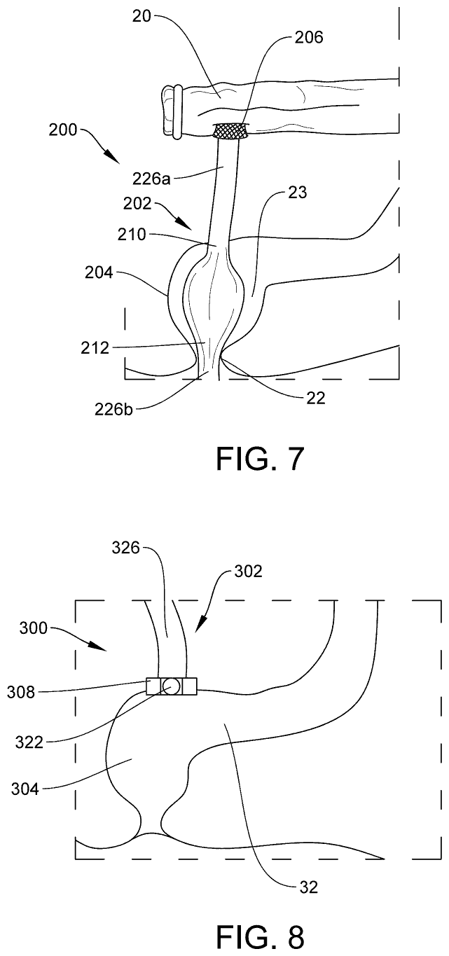

FIG. 7 shows a side view of a bag device of a system according to another exemplary embodiment of the present disclosure;

FIG. 8 shows a side view of a portion of a bag device of a system according to another exemplary embodiment of the present disclosure; and

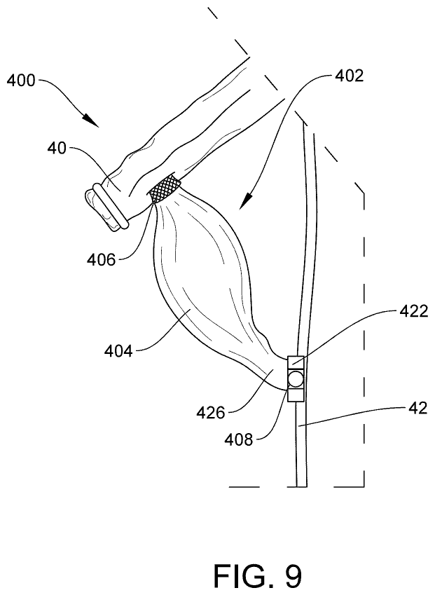

FIG. 9 shows a side view of bag device of a system according to yet another exemplary embodiment of the present disclosure.

### DETAILED DESCRIPTION

The present disclosure may be further understood with reference to the following description and the appended drawings, wherein like elements are referred to with the same reference numerals. The present disclosure relates to an ostomy system and, in particular, relates to an internal ostomy device configured to facilitate a collection of waste from, for example, the digestive or urinary tracts. Exemplary embodiments of the present disclosure comprise an ostomy device including a bag or reservoir configured to be placed internally to eliminate the use of external bags.

A first end of the bag is connected to an end of a target organ (e.g., the small or large intestine or the ureters) while a second end of the bag is connected to a desired exit location via which waste collected in the bag may be evacuated from the body. The ostomy device is configured to collect and hold waste contents until evacuation is possible, and includes a sensor to detect when the ostomy device is required to be emptied. Although the exemplary embodiments describe colostomy procedures in which the end of a colon is attached to a stoma formed in the abdominal wall, it will be understood by those of skill in the art that the systems and methods of the present disclosure may be utilized for any ostomy procedure, including ileostomies in which an end of the small intestine is re-routed to an abdominal stoma and urostomies in which a portion of small intestine is resected and coupled between the ureters and an abdominal stoma.

As shown in FIGS. 1-5, an ostomy system 100 according to an exemplary embodiment of the present disclosure comprises a bag device 102, which may be internally placed within a living body via an endoscopic or laparoscopic procedure utilizing, for example, a delivery device 130 or other delivery device. According to an exemplary embodiment, the bag device 102 includes an expandable reservoir 104 configured to collect waste (e.g., from the intestines) therein, a first connector 106 configured to connect a first end 110 of the reservoir 104 to an end of a target organ (e.g., a large organ intestine 10), and a second connector 108 configured to connect a second end 112 of the reservoir 104 to an exit location 12 such as, for example, an opening in an abdominal wall (e.g., stoma), an anus, or a sigmoid colon.

In an exemplary embodiment, the reservoir 104 is formed of an expandable material and is configured to collect and hold contents flowing from the end of the target organ (intestine 10) until, for example, the reservoir 104 is required to be emptied and/or a user (e.g., ostomy patient) is ready to empty the reservoir 104. The bag device 102 may additionally include a sensor 114 positioned along, for example, a portion of the reservoir 104. The sensor 114 of such an embodiment is configured to indicate to the user of the bag device 102 that the reservoir 104 is full and/or should be emptied.

The reservoir 104 of the bag device 102 extends from the first end 110 to the second end 112 and defines an interior space within which waste may be received and collected until it is desired and/or necessary to empty the bag device 102. In an exemplary embodiment, waste may be received within the reservoir 104 from the target organ via the first end 110 and stored within the reservoir 104 until evacuation of the reservoir 104 is desired. The user may then empty the reservoir via the second end 112. In an exemplary embodiment, the bag device 102 and, in particular, the reservoir 104, is formed of a biocompatible and expandable material such as, for example, silicone, which expands as waste is received and collected therein. The material of the reservoir 104 may, optionally, have elastomeric properties so that the reservoir 104 may deform and expand as waste is received therein, but reverts to an initial, unexpanded shape as contents are drained and/or emptied therefrom.

The first end 110 of the reservoir 104 is connected to the first connector 106, which is configured to be connected to an end of the target organ (intestine 10) so that the space within the reservoir 104 is in communication with an internal space of the target organ so that material flowing through the target organ passes into the reservoir 104. In an exemplary embodiment, the first connector 106 is self-expanding so that, as the first connector 106 is inserted into an opening 14 extending through a wall 16 of the intestine 10 (formed, e.g., via a puncturing device 140 such as, for example, an electrocautery device) the first connector 106 expands to engage the opening 14, thereby fixing the reservoir 104 relative to the intestine 10.

According to an exemplary embodiment, the first connector 106 includes an expandable mesh coil 116 or tube substantially similar to a stent extending out of the end of the first connector 106 so that, when the first connector 106 is inserted into the opening 14, the mesh coil 116 extends into the internal space of the target organ and, after insertion of the first connector 106 into the interior of the target organ, expands within the target organ to a configuration in which at least one cross-sectional dimension of the mesh coil 116 is larger than a diameter of the opening 14.

Specifically, the mesh coil 116 of an exemplary embodiment includes a first expandable flange 118 configured to be positioned within the target organ to engage an interior wall 18 of the intestine 10 and a second expandable flange 120 configured to remain outside the target organ to engage an exterior wall of the target organ (e.g., the intestine 10) to anchor the first connector 106 within the opening 14. Thus, a position of the first connector 106 relative to the wall 16 of the intestine 10 may be fixed and the likelihood of inadvertent disengagement of the bag device 102 from the target organ (e.g., the intestine 10) is reduced.

In an exemplary embodiment, the first connector 106, including the first and second flanges 118, 120, is biased toward the expanded configuration so that, when constrained within a delivery device, the first connector 106 is held in a compressed configuration. As described below the delivery device 130 may be inserted into the target organ via the opening 14 and the first flange 118 is extended distally out of the delivery device 130. At this point, the first flange 118 expands radially outward under its natural bias so that its diameter is greater than the diameter of the opening 14. This prevents the first connector from being pulled out of the target organ.

The user then pulls the delivery device 130 and the first connector 106 proximally to seat the first flange 118 against the inner wall of the target organ. At this point, the user withdraws the delivery device 130 proximally while holding the first connector 106 in place. Thus, the delivery device 130 is moved proximally off of the second flange 120 and the second flange 120 expands radially outward to prevent movement of the first connector distally into the target organ. Thus, the position of the first connector 106 is substantially fixed relative to the target organ and the opening 14.

While the first and second flanges 118, 120 are sufficient to fix the first connector 106 to the target organ or intestine 10 in most cases, in some embodiments, it may be desirable to use an another connecting mechanism for connecting the first connector 106 to the target organ. For example, when used for a urostomy procedure, given the size and fragility of a ureter, the first connector 106 may be sutured to the ureter and/or connected to the ureter via an adhesive. In an alternate embodiment, the bag device 102 may be configured to create an anastomosis directly into a renal pelvis.

As the first end 110 of the reservoir 104 is connected to the first connector 106, the reservoir 104 is locked in a desired position relative to the target organ with an interior of the reservoir 104 fluidly coupled to the interior space of the target organ via the opening 14. Specifically, the distal end of the reservoir 104 is preferably coupled to the first flange 118 so that the distal end of the reservoir 104 is held in place within the target organ so that contents of the target organ flow into the distal end of the reservoir 104 that is within the target organ and out through the opening into more proximal portions of the reservoir 104.

As indicated above, the bag device 102 is inserted into and positioned at a target location (e.g., with a distal end thereof within the intestine) within the body via a delivery device 130 which may include, for example, a catheter. It will be understood by those of skill in the art, however, that the delivery device 130 may have any of a variety of configurations so long as the delivery device 130 extends longitudinally and includes a lumen or channel extending therethrough sized and shaped to permit the bag device 102 to be inserted to a target location (e.g., the end of the intestine 10) within the living body.

In particular, the bag device 102 may be inserted through the delivery device 130 to the target location, in an unexpanded configuration, in which the first and second flanges 118, 120 are constrained in the unexpanded configuration via an interior surface (e.g., interior diameter) of the lumen of the delivery device 130. Upon insertion of a distal end 132 of the delivery device 130 through, for example, the opening 14 and into the intestine 10, the bag device 102 is moved distally relative to the delivery device 130 so that the first flange 118 extends distally past a distal end 132 of the delivery device 130.

As the first flange 118 is moved distally out of the delivery device 130, the first flange 118 is deployed and permitted to expand to engage the opening 14. The first flange 118 is then withdrawn proximally until it is positioned to abut against the interior wall 18 of the intestine 10, and the delivery device 130 is drawn proximally relative to the bag device 102 until the second flange 120 is moved distally past the distal end 132 of the delivery device 130 and permitted to expand under its natural bias to the biased, expanded configuration in which a diameter of the second flange 120 is greater than a diameter if the opening 14 so that contact between the second flange 120 and the exterior wall of the intestine 10 anchors the first connector 106 and the bag device 102 in a desired position in which the distal end of the bag device 102 is open to the interior of the target organ. Thus, a portion of the wall 16 of the intestine 10 is fixed between the first and second flanges 118, 120 of the first connector 106 so that the first end of the reservoir 104 is fixedly connected to the intestine 10.

The second end 112 of the reservoir 104 is connected to the second connector 108, which is configured to be connected to the exit location 12. In an exemplary embodiment, the second connector 108 of an exemplary embodiment includes a control element 122 configured to control the emptying of the reservoir 104. In an exemplary embodiment, the control element 122 includes a valve 124 in communication with the interior space of the reservoir 104. The valve 124 of one exemplary embodiment is configured to be moved between a closed configuration, in which fluid is prevented from flowing therepast, and an open configuration, in which fluid is permitted to flow therethrough. Thus, when the valve 124 is in the closed configuration, the waste collected within the reservoir 104 is prevented from flowing out of the reservoir 104. When the valve 124 is moved toward the open configuration, however, the reservoir 104 is open to the stoma so that the reservoir may be emptied.

As would be understood by those skilled in the art, the valve 124 may take any of a number of known configurations. In an exemplary embodiment, the valve 124 includes a known pressure-controlled valve configured to be moved from the open configuration to the open configuration when a pressure within the reservoir 104 exceeds a predetermined threshold pressure. Thus, an overload of the reservoir 104 is prevented by automatic venting through the valve 124. In another embodiment, the valve 124 may be moved between the open and closed configurations via a user input (e.g., by manipulation of the valve 124 or an actuator coupled thereto by the user).

Alternatively or in addition, as will be described in further detail below, the valve 124 may be automatically moved from the closed configuration to the open configuration when the sensor 114 indicates that the reservoir is full and/or should be emptied. As would be understood by those skilled in the art, a default position of the valve 124 will be the closed configuration so that waste is not released from the reservoir 104 in a manner not desired by the patient. The only exception to this rule will be that the valve 124 may be configured to open when conditions indicate that failure to open the valve 124 may be dangerous to the patient (e.g., if there is a danger that the reservoir 104 will fail if the valve 124 is not opened).

In another embodiment, the control element 122 includes an artificial sphincter rather than a valve 124. The artificial sphincter may include, for example, an inflatable member which is moved between an inflated configuration and a deflated configuration. In the inflated configuration, the inflatable member expands, for example, radially inwardly, to close the exit location 12, thereby preventing waste collected within the reservoir 104 from passing therethrough. When the inflatable member is moved to the deflated configuration, the volume of the inflatable member is reduced to open the exit location 12, allowing the waste to pass therethrough.

It will be understood by those of skill in the art, however, that the artificial sphincter may have any of a variety of configurations so long as the artificial sphincter is configured to open and close the exit location 12, as desired. It will also be understood by those of skill in the art that the control element 122 may have any of variety of configurations and may include any of a variety of mechanisms so long as the control element 122 is configured to control the emptying and/or drainage of the reservoir 104 in a manner conducive to the safety and the concerns of the patient.

It will be understood by those of skill in the art that the second connector 108 may be connected to the exit location via any of a number of configurations depending on the exit location 12 (e.g., anus, stoma, or sigmoid colon) and a configuration of the control element 122 (e.g., valve or artificial sphincter). In one embodiment, the second connector 108 may include features such as, for example, suture holes, configured to facilitate a suturing thereof to tissue adjacent to the exit location 12. In another embodiment, the second connector 108 may be configured to be connected to the exit location via an adhesive (e.g., glue).

In another embodiment, the second connector 108 includes a self-expanding component substantially similar to the mesh coil 116 described above with respect to the first connector 106. In yet another embodiment, the second connector 108 may include protrusions such as, for example, barbs, which are configured to extend into a surrounding tissue to anchor the bag device 102 at the exit location. In another exemplary embodiment, the second connector 108 may be formed of, include, and/or may be coated with an extracellular matrix material that induces ingrowth of cells to facilitate incorporation of the second connector 108 at the exit location 12. Examples of such an extracellular matrix material may include, for example, collagen, fibrin, or blends of different biologic materials or fibrous synthetic material such as polylactic acid (PLA), polylactic-co-glycolic acid (PLGA) and/or other absorbable or non-absorbable materials that encourage tissue ingrowth. In an alternate embodiment, the second connector 108 may facilitate a passage thereof through the exit location 12 so that the control element 122 extends exterior to the user body. As would be understood by those skilled in the art, any combination of these features may be included in the second connector 108 (e.g., suture holes, one or more expandable flanges, barbs, extracellular matrix materials) to achieve a desired fixation or a desired set of fixation options.

In some embodiments, as will be described in further detail below, depending on a desired placement of the bag device 102 and, in particular, the reservoir 104, in the living body in an operative configuration, the first connector 106 may be connected to the first end 110 and the second connector 108 may be connected to the second end 112 of the reservoir 104 via tubing 126. A length of the tubing 126 that separates the first and second connectors 106, 108 from the first and second ends 110, 112, respectively, generally corresponds to a distance between the desired placement locations for the first and second ends 110, 112 of the bag device 102 when the bag device 102 within the living body.

For example, in the exemplary embodiment shown in FIG. 1, the bag device 102 is configured to connect an end of the intestine 10 to an exit location 12 at the anus. In this embodiment, the first connector 106 is connected directly to the first end 110 of the reservoir 104 while the second connector 108 is connected to the second end 112 of the reservoir 104 via the tubing 126. Thus, the bag device 102 may be placed within the user body with the reservoir 104 positioned outside an intestinal tract while the tubing 126 extends through a rectum to the anus so that the second connector 108 may be connected thereto. It will be understood by those of skill in the art that the tubing 126 is configured to accommodate the passage therethrough of the intestinal contents as the reservoir 104 is being drained. Thus, while the tubing 126 should also be formed of a biocompatible material, the tubing 126 is not configured to expand to store intestinal contents therein.

In an exemplary embodiment, the sensor 114 is positioned at a location along a portion of the reservoir 104 and is configured to detect a level of fill of the reservoir 104 and/or to provide a signal based on this detected fill level indicating that the reservoir should be emptied. The sensor 114 may be wirelessly connected to, for example, a smartphone or other computing device so that the sensor 114 may communicate to the user the level of fill of the reservoir 104 and/or to indicate whether the reservoir 104 should be emptied (or that the reservoir 104 may soon need to be emptied) via an application stored on the smartphone or computing device.

In an exemplary embodiment, the sensor 114 may be configured to detect a level of fill in the reservoir 104 by detecting a pressure within the reservoir 104 corresponding to the level of fill thereof. For example, when the pressure detected by the sensor 114 exceeds a predetermined threshold pressure, the sensor 114 may generate a signal that indicates to the user that the bag device 102 should be emptied. The indication may be configured as, for example, an auditory and/or visual alert.

In another exemplary embodiment, the sensor 114 is configured to detect deformation of the reservoir 104 (i.e., a change in a shape and/or size of the reservoir 104 or of a particular dimension of the reservoir 104) to determine the level of fill and/or whether the reservoir 104 should be emptied. As will be understood by those of skill in the art, the reservoir 104 deforms as it expands to accommodate waste received therein. Thus, the deformation of the reservoir 104 corresponds to the fill level of the reservoir 104. In an exemplary embodiment, when the deformation of the reservoir 104 exceeds a predetermined level, an alert may be provided to the user indicating that the reservoir 104 should be emptied. It will be understood by those of skill in the art, however, that the sensor 114 may have any of a variety of configurations so long as the sensor 114 is configured to detect the fill level of the reservoir.

According to an exemplary method utilizing the system 100, the bag device 102 is implanted at a desired location within a living body using, for example, an endoscopic or laparoscopic procedure. According to the example shown in FIG. 1, in which the first connector 106 is connected to the end of the intestine 10 and the second connector 108 is connected to the exit location 12 at the anus, the puncturing device 140 (e.g., electrocautery device) may be inserted through the anus and rectum to puncture a first hole via, for example, cauterization, through a wall thereof to exit the intestinal tract. A second hole may be created through the wall 16 (e.g., via a puncture from an exterior surface to an interior surface of the wall 16) at the end of the intestine 10 to form the opening 14.

The delivery device 130, or other similar delivery device, within which the bag device 102 is received during the delivery process, is then inserted through the anus, along a path formed via the puncturing device 140, until the distal end 132 of the delivery device 130 extends through the opening 14 into the interior of the end of the intestine 10. The first connector 106 is then deployed to engage the opening 14 by, for example, drawing the delivery device 130 proximally relative to the bag device 102 as described above to expand the first and second flanges 118, 120 against the interior and exterior walls of the intestine 10 as described above so that the mesh coil 116, the bag device 102 and the first connector 106 are fixed relative to the opening 14.

Upon engagement of the first connector 106 with the end of the intestine 10, the delivery device 130 may be drawn further proximally relative to the bag device 102 so that the reservoir 104 is positioned externally of any portion of the intestinal tract, and the tubing 126 connecting the reservoir 104 to the second connector 108 is received within the rectum. The second connector 108 may then be connected to the exit location 12 at the anus so that the control element 122 is positioned proximate thereto. Thus, as described above, intestinal contents flowing from the end of the intestine 10 may be collected within the reservoir 104 until it is desired to empty/drain the reservoir—e.g., when the sensor 114 indicates that the reservoir should be emptied or when the user has access to a toilet. When it is desired to empty the reservoir 104, the user may, in an exemplary embodiment, remotely control the control element 122 to move from the closed configuration to the open configuration.

According to an alternate method of placement of the system 100, as shown in FIG. 6, the first connector 106 of the bag device 102 is connected to the end of the intestine 10, as described above. Rather than connecting the second connector 108 to the anus so that the control element 122 is positioned internally—i.e., within the living body—the tubing 126 is passed through the rectum and the anus so that the control element 122 is located along an exterior of the living body.

As described above, the system 100 may be utilized in a variety of configurations with the bag device 102 placed in a variety of positions within the user body—e.g., at varying exit locations 12. The exemplary embodiments shown in FIGS. 7-9 and described below, show examples of bag devices 102 placed such that second connectors 108 thereof are positioned at varying exit locations of the living body. It will be understood by those of skill in the art, however, that the placement and/or configuration of the bag devices described herein are non-limiting and that the bag devices of the present disclosure may have any of a variety of configurations and/or placements within a user body so long as a reservoir thereof is internally placed within the user body and configured to be drained/emptied as necessary and/or desired. It will also be understood by those of skill in the art that the bag devices described below may be substantially similar to the bag device 102, except as indicated below.

As shown in FIG. 7, a system 200 according to another exemplary embodiment may be substantially similar to the system 100 described above, comprising a bag device 202 including a first connector 206 configured to be connected to an end of a target organ (e.g., an intestine 20) and a reservoir 204 configured to receive and collect waste from the target organ therein until the user is ready to empty/drain the reservoir 204. The reservoir 204 of one exemplary embodiment, however, is configured to be received within a lumen of, for example, the sigmoid colon 23 so that a control element of a second connector (not shown) extends through the anus 22 to an exterior of the user body.

In this embodiment, the first connector 206 may be connected to a first end 210 of the reservoir 204 via a first tubing 226A, a length of which is selected to facilitate receipt of the reservoir 204 in, for example, the sigmoid colon. Similarly to the first connector 106 of the bag device 102, the first connector 206 may be configured to be connected to an end of a target organ such as the intestine 20 and may include, for example, a self-expanding mesh coil.

In an exemplary embodiment, the second connector is connected to a second end 212 of the reservoir 204 via a second tubing 226B, a length of which is selected to allow passage of the second tubing 226B through the anus so that the second connector may be positioned external to the living body when the first connector 206 is positioned in the target organ as desired. It will be understood by those of skill in the art that the bag device 202 may be positioned, as described, using a method substantially similar to the method described above with respect to the system 100.

According to an alternate embodiment, the bag device 202 may be similarly positioned such that the reservoir 204 is received within the sigmoid colon, but with the second connector proximate the anus, within the living body.

As shown in FIG. 8, a system 300 according to another exemplary embodiment, is substantially similar to the systems 100, 200 described above, comprising a bag device 302 including a first connector (not shown) configured to be connected to an end of a target organ such as an intestine 30. Similarly to the bag device 102, a reservoir of the bag device 302 is configured to be received internally within a living body external to the target organ (i.e., external to the intestinal tract). Rather than a second connector 308 connected to the anus, however, the second connector 308 of the bag device 302 is connected directly to a portion of the colon 32—e.g., proximate the sigmoid colon or the rectum-so that intestinal contents collected within the reservoir 304 are emptied into natural lumens of the intestinal tract and rely on natural means to be expelled from the user body. As will be understood by those of skill in the art, the bag device 302 of the system 300 may be particularly useful for instances in which the patient has functioning organs—e.g., a functioning anal sphincter so that the patient can control evacuation of waste in the natural manner.

In an exemplary embodiment, the second connector 308 is directly coupled to a second end of the reservoir (not shown) or, alternatively, may be connected to the reservoir via tubing 326. The second connector 308 may include, for example, a self-expanding mesh coil substantially similar to the mesh coil described above with respect to the first connector and may be configured to be directly coupled to a portion of the target organ (e.g., the colon 32)—i.e., without a control element so that the patient controls the evacuation of waste in a natural manner (e.g., by control of the anal sphincter). In another embodiment, the second connector 308 includes a control element 322 configured to control the flow of waste therethrough from the reservoir (not shown) into a downstream portion of the colon 32. The control element 322 may include a valve such as, for example, a gating valve as described above.

As shown in FIG. 9, a system 400 may be substantially similar to the systems 100-300 described above, comprising a bag device 402 including a first connector 406 connected to an end of a target organ such as an intestine 40 with a reservoir 404 configured to receive and collect waste flowing from the target organ. A second connector 408 is connected to an exit location 42 configured as an opening extending through an abdominal wall to form a stoma. In this embodiment, the second connector 408 is configured to engage the opening in the abdominal wall via any one of a variety of connecting mechanisms (e.g., expanding mesh, suturing) as described above in regard to the previous embodiments and may include a control element 422 substantially similar to the control element 122 described above with respect to the bag device 102. For example, the control element 422 may include a valve or an artificial sphincter.

Although the exemplary embodiment shows and describes the second connector 408 connected to the opening of the abdominal wall, it will be understood by those of skill in the art that, in another embodiment, tubing 426 may connect the second connector 408 to the reservoir 404 with the tubing 426 being passed through the opening of the abdominal wall so that the control element 422 extends exterior to the living body.

It will be apparent to those skilled in the art that various modifications may be made in the present disclosure, without departing from the scope of the disclosure. Furthermore, those skilled in the art will understand that the features of any of the various embodiments may be combined in any manner that is not inconsistent with the description and/or the functionality of the embodiments.

1-15. (canceled)

16. An ostomy bag device, comprising:
an expandable reservoir extending from a first end to a second end and defining a space therewithin;
a first connector connected to the first end of the reservoir and configured to engage an opening extending through a wall at an end of an intestine so that intestinal contents flowing from the end of the intestine are received and collected within the reservoir; and
a second connector connected to the second end of the reservoir and configured to engage an exit location via which contents collected within the reservoir are to be drained to an exterior of a patient body, the second connector including a control element movable between a closed configuration, in which contents of the reservoir are prevented from flowing therepast, and an open configuration, in which contents of the reservoir are permitted to flow therepast to drain the reservoir.

17. The device of claim 16, further comprising:
a sensor positioned along the reservoir and configured to detect a level of fill thereof.

18. The device of claim 17, wherein the sensor includes one of a pressure sensor and a deformation sensor.

19. The device of claim 16, wherein one of the first connector and the second connector includes a self-expanding mesh coil.

20. The device of claim 19, wherein the self-expanding mesh coil includes a first flange at a first end thereof and a second flange at a second end thereof, the first and second flanges configured to receive a portion of the wall of the intestine therebetween.

21. The device of claim 16, wherein one of the first and second connectors is connected to the first and second ends of the reservoir, respectively, via a tubing.

22. The device of claim 16, wherein the control element includes one of a valve and an artificial sphincter.

23. The device of claim 16, wherein the reservoir is formed of a biocompatible and expandable material.

24. The device of claim 23, wherein the biocompatible and expandable material includes silicone.

25. The device of claim 16, wherein the reservoir is deformable from an initial, unexpanded configuration toward an expanded configuration as the intestinal contents are received therein.

26. A system, comprising:
a bag device including an expandable reservoir, a first end of the expandable reservoir configured to be connected to an end of an intestine via a first connector so that intestinal contents flowing from the end of the intestine are received and collected within the expandable reservoir, and a second end of the expandable reservoir configured to be connected to an exit location via a second connector so that the intestinal contents collected within the expandable reservoir are to be drained to an exterior of a patient body, the second connector including a control element movable between a closed configuration, in which contents of the reservoir are prevented from flowing therepast, and an open configuration, in which contents of the reservoir are permitted to flow therepast to drain the expandable reservoir; and
a delivery device extending longitudinally and including a channel extending therethrough, the bag device insertable through the channel to a target location within a user body.

27. The system of claim 26, further comprising:
a puncturing device configured to form an opening through a wall of the intestine to which one of the first and second connectors of the bag device is to be engaged.

28. The system of claim 26, wherein the bag device includes a sensor positioned along the reservoir, the sensor configured to detect a fill level of the reservoir.

29. The system of claim 26, wherein one of the first connector and the second connector includes a self-expanding mesh coil.

30. The system of claim 26, wherein the control element includes one of a valve and an artificial sphincter.

31. A method for treating an intestine, comprising:
engaging a first connector connected to a first end of a reservoir with an opening extending through a wall at an end of the intestine so that intestinal contents flowing from the end of the intestine are received and collected within the reservoir; and
engaging a second connector connected to a second end of the reservoir to an exit location, the second connector including a control element movable between an open configuration, in which contents of the reservoir are permitted to flow through the control element, through the exit location, to an exterior of a user body, and a closed configuration, in which contents of the reservoir are prevented from flowing through the control element.

32. The method of claim 31, further comprising:
detecting a level of fill of the reservoir to determine whether to drain the reservoir.

33. The method of claim 31, wherein the exit location includes one of an anus and an opening extending through an abdominal wall of the user body.

34. The method of claim 31, wherein engaging the first connector with the opening extending through the wall of the intestine includes deploying a self-expanding mesh coil.

35. The method of claim 31, further comprising:
forming the opening through the wall at the end of the intestine via an electrocautery device.
